(19) United States
(12) Patent Application Publication
(10) Pub. No.: US 2025/0160715 A1
(43) Pub. Date: May 22, 2025

(54) EEG SIGNAL DETECTING HEADBAND

(71) Applicant: Zeit Medical, Inc., Redwood City, CA (US)

(72) Inventors: Orestis Vardoulis, San Francisco, CA (US); Urs H. Naber, Redwood City, CA (US)

(21) Appl. No.: 18/952,797

(22) Filed: Nov. 19, 2024

Related U.S. Application Data
(60) Provisional application No. 63/601,480, filed on Nov. 21, 2023.

(51) Int. Cl. A61B 5/256; A61B 5/00; A61B 5/291; A61B 5/369

(52) U.S. Cl. CPC A61B 5/256; A61B 5/291; A61B 5/369; A61B 5/6831; A61B 2562/164

(57) ABSTRACT

An EEG signal monitoring headband can include one or more features that can help provide improved EEG signal acquisition. Such improved EEG signal acquisition can, in turn, help provide better detection and diagnosis of stroke or one or more other conditions of diagnostic interest. Features can include temporal electrodes that can project downwardly and inwardly from a main body portion of the headband, such as to locate the temporal electrodes against corresponding mastoid process or other target locations of the subject. The headband can include fore and aft portions that engage each other via clips. This can help allow one-handed user-adjustment of the headband length, temporal electrode position, or the like.

### CLAIM OF PRIORITY

This application claims the benefit of priority of U.S. Provisional Patent Application No. 63/601,480, filed Nov. 21, 2024 entitled “EEG SIGNAL DETECTING HEADBAND,” (Attorney Docket No. 5888.004PRV), which is hereby incorporated herein by reference in its entirety.

### CROSS-REFERENCE TO RELATED PATENT DOCUMENTS

This patent application is related to PCT/US2023/023848 entitled “STROKE MONITOR,” which is incorporated by reference herein in its entirety.

### TECHNICAL FIELD

This document pertains generally, but not by way of limitation, to EEG signal monitoring, such as for stroke detection, prediction, or monitoring, and more particularly, but not by way of limitation, to an EEG signal detecting headband.

### BACKGROUND

Intrinsic electrical brain activity can be monitored using an EEG signal detector, which can include electrodes and signal-processing circuitry that can be located on a headband.

### SUMMARY/OVERVIEW

The present inventors have recognized, among other things, that challenges to EEG signal monitoring using a headband can include poor contact between a signal acquisition electrode and a desired target location on the subject's head. An EEG signal detecting headband can be subject to challenges, such as motion of the subject during sleep, the presence of hair interfering with providing good contact between a signal electrode and a target skin location on the patient's head. A comfortable and easy-to-use and easy-to-adjust EEG signal detecting headband can be desirable. But patients' head size, morphology, and other characteristics can vary widely. A patient may also have physical limitations on use of one or both hands, which can make it highly desirable to provide a device or kit that can be easily placed, worn, and adjusted, such that electrodes are properly and securely located so as to provide strong and robust EEG signal monitoring.

This document describes, among other things, an improved EEG signal monitoring headband, and methods of making and using the same. The EEG signal monitoring headband can include one or more features that can help provide improved EEG signal acquisition. Such improved EEG signal acquisition can, in turn, help provide better detection and diagnosis of stroke or one or more other conditions of diagnostic interest. Features can include temporal electrodes that can project downwardly and inwardly from a main body portion of the headband, such as to locate the temporal electrodes against corresponding mastoid process or other target locations of the subject. The headband can include fore and aft portions that engage each other via clips. This can help allow one-handed user-adjustment of the headband length, temporal electrode position, or the like.

This Summary/Overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A (front view), 1B (top view), and 1C (rear view) show an illustrative example of a human wearable EEG monitoring headband.

FIGS. 2A, 2B, and 2C show perspective views of portions of the headband,

FIGS. 3A, 3B, 3C, and 3D show various views of examples of loop-back portions, corresponding pouches, and bent resilient biasing member inserts.

FIG. 4A is a top view showing an example of the fore portion of the headband.

FIG. 4B is a detail view of the region 400 shown in FIG. 4A.

FIG. 5 is a front view showing an example of portions of the fore portion of the headband.

FIG. 6 is a rear view showing an example of portions of the fore portion of the headband.

FIGS. 7A, 7B, 7C, and 7D show various views of an example of portions the cradle assembly, such as in various states of assembly.

FIG. 8A shows a perspective view of an example of an flexible printed circuit (FPC), which can include a unitary piece extending from FPC ends located near the temporal electrode assemblies.

FIG. 8B shows a cutaway view of portions of the headband showing the FPC located therein.

### DETAILED DESCRIPTION

This document describes, among other things, an improved EEG signal monitoring headband, and methods of making and using the same. The EEG signal monitoring headband can include one or more features that can help provide improved EEG signal acquisition. Such improved EEG signal acquisition can, in turn, help provide better detection and diagnosis of stroke or one or more other conditions of diagnostic interest. Features can include temporal electrodes that can project downwardly and inwardly from a main body portion of the headband, such as to locate the temporal electrodes against corresponding mastoid process or other target locations of the subject. The headband can include fore and aft portions that engage each other via clips. This can help allow one-handed user-adjustment of the headband length, temporal electrode position, or the like.

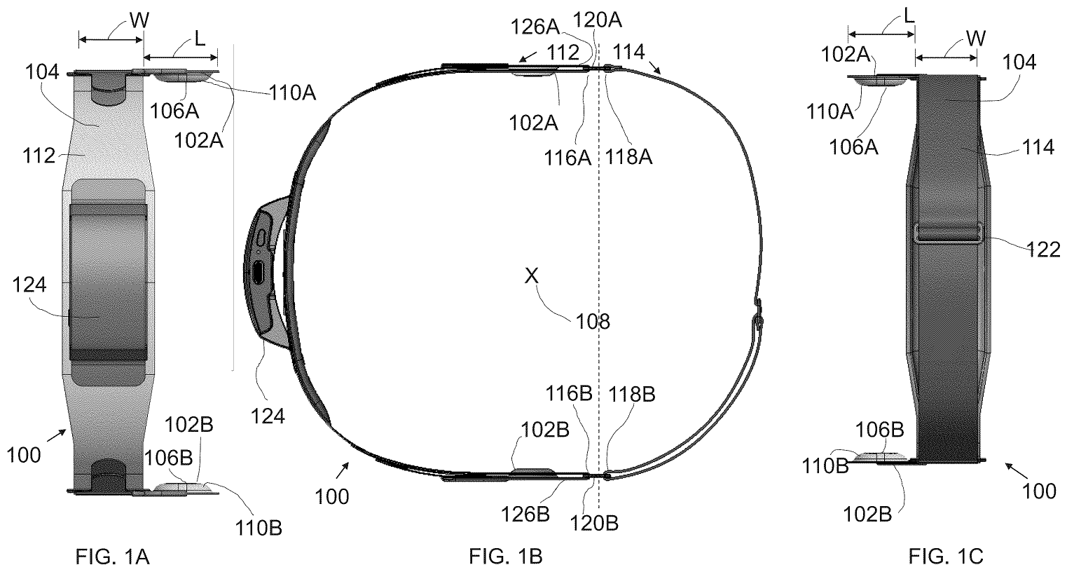

FIGS. 1A (front view), 1B (top view), and 1C (rear view) show an illustrative example of a human wearable EEG monitoring headband 100. The headband 100 can be sized and shaped and otherwise configured to be worn encircling a head of a human subject, such as at a location that is superior to eyes and ears of the subject. The headband 100 can include a pair of temporal electrode assemblies 102A-B. The temporal electrode assemblies 102A-B can protrude inferiorly, such as by a temporal electrode assembly protrusion length “L”, from a main body portion 104 of the headband 100, defining a main body width “W,” as shown. In an example, the inferior-direction protrusions of the temporal electrode assemblies 102A-B permit placement of skin-contacting working surfaces of corresponding electrodes 106A-B against the skin of the subject, such against corresponding mastoid processes of respective temporal regions of the subject. Locating the skin-contacting working surfaces of the electrodes 106A-B against the mastoid processes of respective temporal regions behind (posterior to) the ears of the subject can be helpful to good signal acquisition because, among other things, such mastoid process regions are typically relatively more free of hair that may otherwise come between the electrodes 106A-B and the skin, such as compared to a location that is above (superior to) the ears of the subject, such as on the main body portion 104 of the headband 100.

As explained in more detail herein, the temporal electrode assemblies 102A-B can also be bent, biased, or otherwise directed inwardly toward a central longitudinal axis “X” 108 that can be defined extending in a superior-inferior direction through a longitudinal center of the skull of the subject. This can also help provide good contact for the skin contacting working surfaces of the electrodes 106A-B, such as against the mastoid processes or other desired temporal locations of the subject, since such target regions of the skull may be located closer to the central longitudinal axis 108 due to an inward tapering of the skull inferior to the location at which the main body portion 104 of the headband 100 is worn.

The electrode assemblies 102A-B can include electrode-carrying “pillows” 110A-B or other carriers for the electrodes 106A-B, which can additionally or alternatively help locate the skin-contacting working surfaces of the electrodes 106A-B more closely and firmly against the skin of the skull of the subject, such as at the respective mastoid processes or other desired targeted temporal locations. For example, the respective pillows 110A-B can extend annularly about corresponding individual ones of the electrodes 106A-B that are respectively carried or supported by individual ones of the pillows 110A-B. Thus, the respective pillows 108A-B can optionally be sized, shaped, or otherwise configured to help inwardly locate respective skin-contacting working surfaces of the electrodes 106A-B closer to the central longitudinal axis 108 of the subject than corresponding locations of the main body portion 104 of the headband 100 that are directly superior and adjacent to the electrodes 106A-B or the corresponding electrode assemblies 102A-B, when the headband 100 is being worn on the skull of the subject.

The headband 100 can include a fore portion 112 and an aft portion 114, such as shown in FIG. 1B as being separated by a dashed line. The front view of FIG. 1A shows an example of the fore portion 112 of the headband 100. The rear view of FIG. 1C shows an example of the aft portion 114 of the headband 100. The fore portion 112 of the headband 100 can be sized and shaped and otherwise configured for extending along a forehead region of the subject, such as shown in FIG. 1A, and flexibly bending to extend along each temporal region of the subject, such as shown in FIG. 1B, toward respective fore headband ends 116A-B. The respective fore headband ends 116A-B can be located beyond (posterior to) corresponding temporal electrodes 106A-B and their corresponding electrode assemblies 102A-B. The fore portion 112 of the headband 100 can include an electronics unit cradle assembly 124 or other carrier, such as described in further detail herein. The fore portion 112 of the headband 100 can include loop-back portions 126A-B, such as respectively looped through corresponding engagement clips 120A-B at the corresponding fore headband ends 116A-B and extending back in an anterior direction therefrom.

The aft portion 114 of the headband 100 can be sized and shaped and otherwise configured for extending along a rear skull region of the subject and flexibly bending to extend toward and terminate at respective aft headband ends 118A-B. The aft headband ends 118A-B can respectively be coupled to corresponding fore headband ends 116A-B by corresponding engagement clips 120A-B. The aft portion 114 of the headband 100 can be made of elastic nylon or other stretchable material, such as which can be looped and coupled to from a length-adjustment clip 122 to allow a length of the aft portion 114 of the headband 100 to be user-adjusted. In an example, an adjustable length portion looped through the length-adjustment clip 122 can be sized and shaped to provide length-adjustability in a range between 100 millimeters and 200 millimeters for the aft portion 114 of the headband 100.

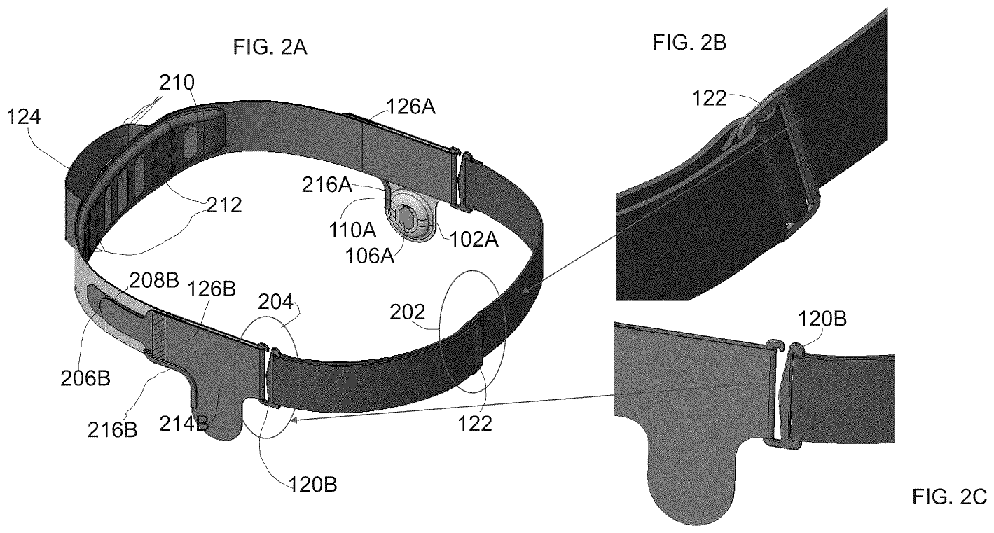

FIGS. 2A, 2B, and 2C show perspective views of portions of the headband 100. FIG. 2A shows a perspective view of the headband 100, including both the fore portion 112 and the aft portion 114. FIG. 2B shows a perspective detail view of the aft portion 114 of the headband 100 in a region 202 around the length-adjustment clip 122. FIG. 2C shows a perspective detail view of a region 204 at which the fore portion 112 and the aft portion 114 of the headband 100 are coupled together by an engagement clip 120. A looped portion of the aft portion 114 of the headband 100 can extend between the engagement clip 120B and the length adjustment clip 122, such as to help provide length adjustment of the aft portion 114 of the headband 100.

FIG. 2A shows an example of the loop-back portions 126A-B of the fore portion 112 of the headband 100, extending back in an anterior direction from corresponding engagement clips 120A-B and terminating at corresponding anterior loop-back ends 206A-B. Hook-and-Loop (e.g., VELCRO®) or other corresponding fasteners 208A-B can be provided between anterior portions of the loop-back portions 126A-B of the fore portion 112 of the headband 100 and underlying non-loop-back portions of the fore portion 112 of the headband 100. The loop-back portions 126A-B and the corresponding fasteners 208A-B can permit the end-user to adjust an effective length of the fore portion 112 of the headband 100, such as in a one-handed manner, such as by detaching a loop-back portion 126A-B of the headband 100 and at least one of pulling the loop-back portion 126A-B of the headband 100 in an anterior direction to tighten the fore portion 112 of the headband 100, or introducing slack into the loop-back portion 126A-B of the headband in a posterior direction to loosen the fore portion 112 of the headband 100. Such user-adjustment can help provide a secure but comfortable fit of the headband 100, to properly locate the temporal electrode assemblies 102A-B such that their corresponding electrodes 106A-B are placed at the desired mastoid process or other desired temporal locations.

FIG. 2A shows an example of various other electrodes 210 that can be included on an interior portion of the fore portion 112 of the headband 100, such as for EEG signal acquisition at their corresponding locations, such as for use in combination with EEG signal acquisition by the temporal electrodes 106A-B. The interior portion of the headband 100 can also include silicone or other gripping protuberances or bumps 212, such as can help stabilize the location of the headband 100 when being worn by the subject.

In FIG. 2A, the loop-back portions 126A-B can respectively include corresponding pouches 214A-B, into which bent resilient biasing member inserts can be insertably placed, such as from a superior direction into an opening of the pouch 214. The pouches 214A-B can extend from the main body 104 portion of the headband 100 downward (in an inferior direction) along the corresponding electrode assemblies 102. Providing bent resilient biasing member stiffening inserts into the corresponding pouches 214A-B can help to further help locate, push, or bias the electrode assemblies 102A-B and their corresponding electrodes 106A-B toward the central longitudinal axis 108 at or toward their desired mastoid process or other temporal locations.

The loop-back portions 126A-B can also respectively include corresponding ear rails or other ear-rest stabilizing members 216A-B, such as which can rest or be seated upon a top and behind an ear of the subject. The ear-rest stabilizing members 216A-B can be made of a stiffer material (e.g., plastic or silicone) than what is used for other portions of the pouches 214 or the loop-back portions 126A-B. These relatively stiffer ear-rest stabilizing members can be located on an inferior and anterior portion of a corresponding individual one of pouches 126A-B, and can help provide a predictable location of the corresponding electrode assemblies 102A-B, which, in turn, can help provide a predictable location of the corresponding electrodes 106A-B such that their respective working surfaces can contact desired mastoid process or other desired temporal skin locations of the subject in a predictable manner.

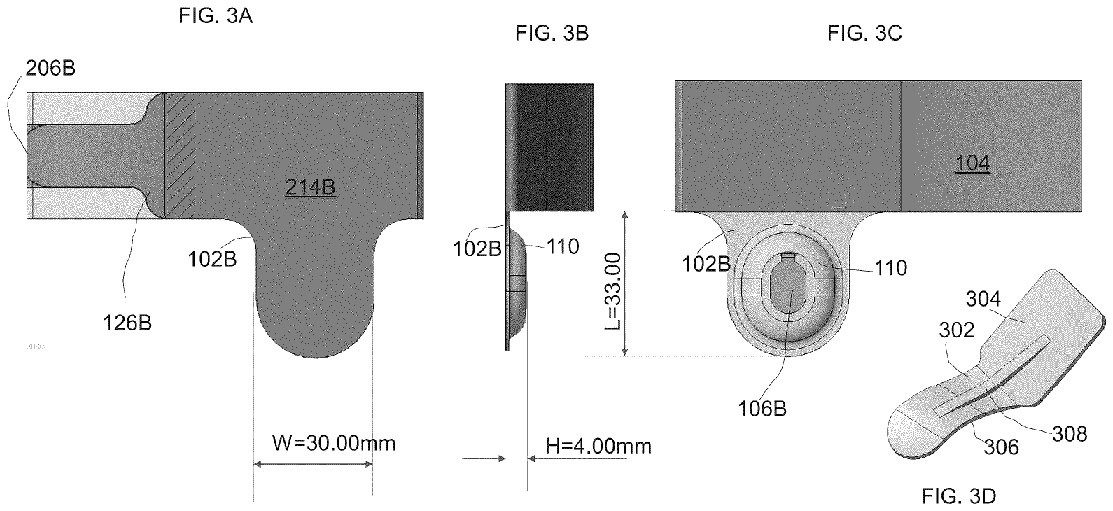

FIGS. 3A, 3B, 3C, and 3D show various views of examples of the loop-back portions 126A-B, corresponding pouches 214A-B, and bent resilient biasing member inserts 302. FIG. 3A shows a side view of an example of a loop-back portion 126B and its corresponding pouch 214 into which a bent insert 302 can be inserted from above. In FIG. 3A, the ear-rest stabilizing member 216B is omitted for clarity, but can optionally be included. FIG. 3A illustrates an example in which a width “W” of the electrode assembly 102B is shown as 30 millimeters. FIG. 3B is a rear view illustrating an example in which a height “H” of the pillow 110 is 4 millimeters. FIG. 3C illustrates a side view looking out from the subject's skull, in which the length “L” of the electrode assembly 102B protrudes by 33 millimeters from a main body 104 portion of the headband 100. FIG. 3D shows an example of a bent insert 302, such as which can be inserted into the pouch 214B of the loop-back portion 126B of the fore portion 112 of the headband 100, such as for biasing or otherwise locating a working surface of the electrode 106B against skin covering a mastoid process or other desired temporal location of the subject. The bent insert 302 can include a superior portion 304, such as which can be located in the pouch 214 against a main body 104 portion of the headband 100, and an inferior portion 306, such as which can be located in the pouch 214 against electrode assembly 102B protruding in an inferior direction from the main body 104 of the headband 100. In this example, the superior portion 304 of the insert 302 can be relatively planar, and the inferior portion 306 of the insert 302 can be bent therefrom and curved, such as to help locate a working surface of the electrode 106B closer to the central longitudinal axis 108 than the main body portion 104 of the headband 100. A bent stiffening rib 308 can be included on the insert 302, such as can extend between the superior portion 304 of the insert 302 and an inferior portion 306 of the insert 302. Multiple pairs of inserts 302 can be provided in a kit or package, such as with different bend amounts, such as to permit the user to select an appropriate one of the inserts, with an appropriate bend, curvature, or both, to provide a desired degree of inward locating of the working surface of a corresponding one of the electrodes 106 against skin covering a mastoid process or other desired temporal location of the subject.

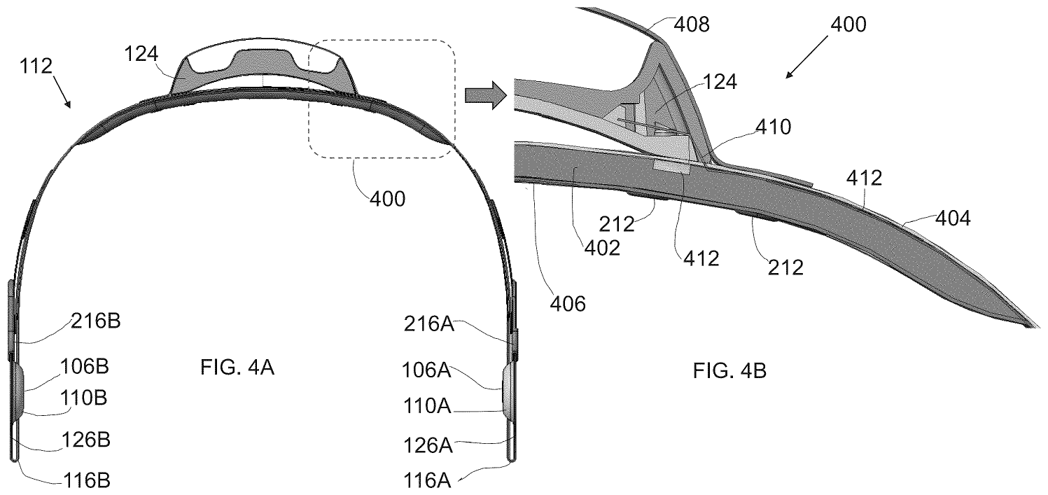

FIG. 4A is a top view showing an example of the fore portion 112 of the headband 100. FIG. 4B is a detail view of the region 400 shown in FIG. 4A. A head foam portion 402 (e.g., 3 millimeters thickness) can be included between the electronics unit cradle assembly 124 and a frontal portion of the fore portion 112 of the headband 100, such as to help provide comfort to the user when worn. The foam portion 402 can be covered by a nylon (e.g., LYCRA®) or other outer fabric 404, located away from the wearer, and an inner fabric 406, located toward the wearer. An outer portion of the cradle assembly 124 can be covered by a band or pouch of heat-stick nylon (e.g., LYCRA®) or other fabric 408, which can additionally help secure an electronics unit within the cradle assembly 124. The electronics unit can perform signal-processing of signals received from various electrodes of the headband, such as described in Vardoulis et al.

PCT/US2023/023848 entitled STROKE MONITOR, filed on May 30, 2023, which is hereby incorporated herein by reference in its entirety, including its description of an electronics unit and signal processing performed by the electronics unit or by an ancillary computing device that can be communicatively coupled to such an electronics unit. A flexible printed circuit (FPC) 410 can extend along a frontal and temporal portions of the fore portion 112 of the headband 100, such as toward the fore ends 116A-B, but terminating at the electrode assemblies 102A-B to provide electrically conductive traces, from corresponding electrical contacts in the cradle assembly 124, that can respectively connect directly or indirectly to corresponding individual ones of the electrodes 106A-B. The cradle assembly 124 can be located in a middle portion of the fore portion of the headband and can include one or more anchors 412, such as which can extend and affix into the foam 402. This can help better secure the cradle assembly 124 to the fore portion 112 of the headband 100. A stiffening or strengthening nylon (e.g., LYCRA®) or other cloth layer 414 can be provided between the foam 402 and the FPC 410.

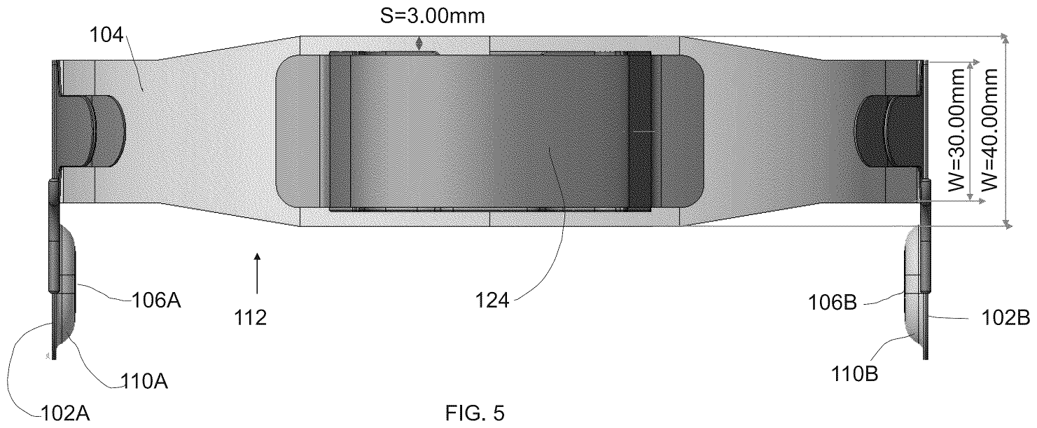

FIG. 5 is a front view showing an example of portions of the fore portion 112 of the headband 100. As shown in FIG. 5, the main body portion 104 of the fore portion 112 of the headband 100 can flare out from a width of W=30 millimeters, such as at a location that is superjacent to the electrode assemblies 102A-B, to a width of W=40 millimeters, such as at a location behind the electronics unit cradle assembly 124 to be worn located on the subject's forehead. This can allow a spacing of about S=3 millimeters between the electronics unit cradle assembly 124 and the adjacent edges of the main body portion 104 of the fore portion 112 of the headband 100.

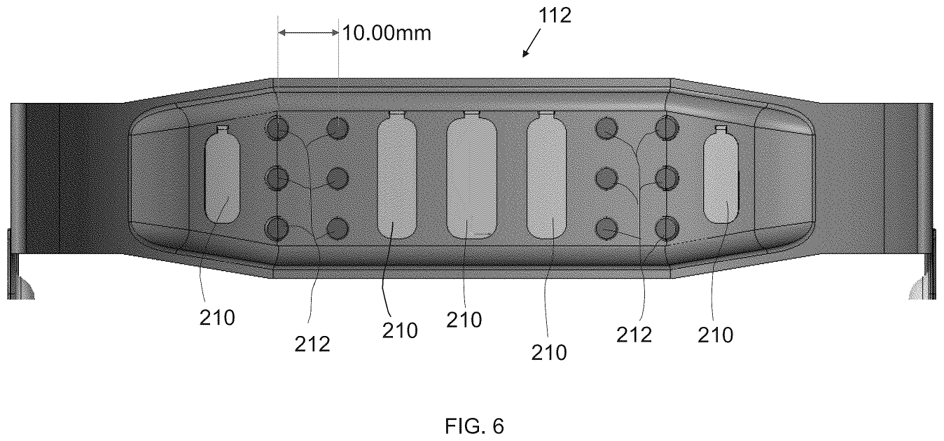

FIG. 6 is a rear view showing an example of portions of the fore portion 112 of the headband 100. As shown in FIG. 6, electrodes 210 and gripping bumps 212 can be located on an interior portion of the fore portion 112 of the headband 100. Electrical connections can extend between the FPC 410 and the electrodes 210, such as through the foam 402 and the inner fabric 406 located therebetween. In the example of FIG. 6, a spacing between vertical columns of gripping bumps 212 can be about 10 millimeters, and a size of the gripping bumps 212 can be about 5 millimeters in diameter with a height of between about 0.3 millimeters and 0.5 millimeters. In the example of FIG. 6, twelve gripping bumps 212 are shown, in an arrangement of 4 columns of three gripping bumps 212. Five electrodes 210 are shown, three of which are arranged to be located centrally on the forehead of the subject, the other two of which are located offset toward opposing lateral sides, and each separated from the three centrally located electrodes 210 by two columns of the gripping bumps 212.

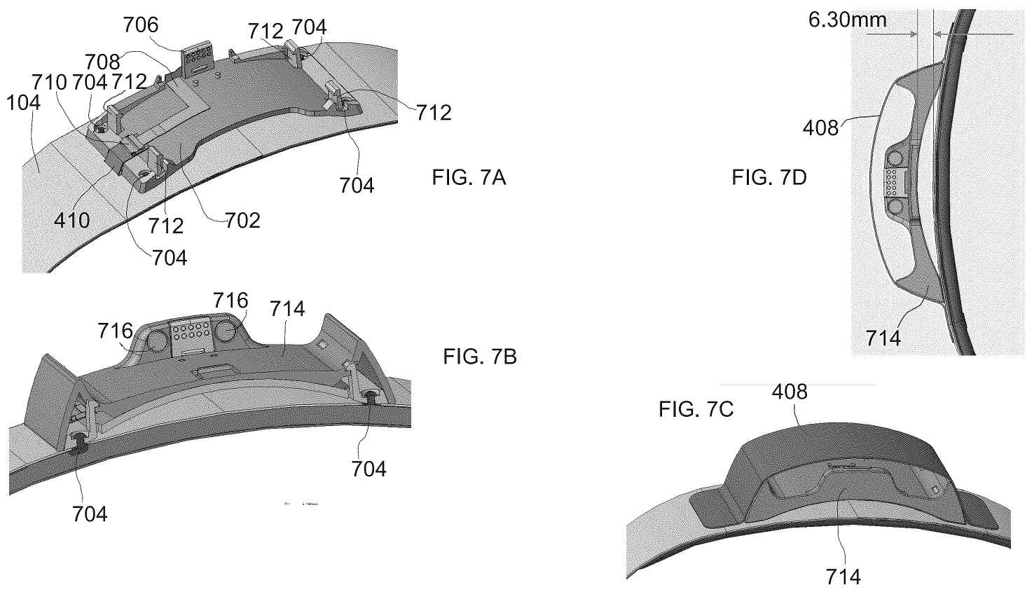

FIGS. 7A, 7B, 7C, and 7D show various views of an example of portions the cradle assembly 124, such as in various states of assembly. In FIG. 7A, a base 702 of the cradle assembly 124 has been affixed to a main body 104 portion of the fore portion 112 of the headband 100, such as by snap-fit rivets 704 having respective base ends being fixedly embedded and anchored within the foam 402 and having respective top ends snap-fitted into the base 702, thereby attaching the base 702 to the main body 104 portion of the fore portion 112 of the headband 100. The base 702 can include a curved underside to be located toward the subject's head, such as to accommodate curvature of the subject's forehead pressed against the foam 402, which, in turn, can press into the curved underside of the base 702. The base can include a multi-contact connector block 706 projecting in an anterior direction at a bottom (e.g., inferior) edge of the base 702, against which a similar arrangement of multiple contacts of an electronics unit can be placed into the cradle assembly 124. The multi-contact connector block 706 can be connected, via an FPC 708 and a connector block 710, to the electrodes 210 and to the temporal electrodes 106 via respective individual electrical traces on the FPC 410. The base 702 can include snap-fit tabs 712, such as for snap-fitting engagement into a electronics unit carrier 714, such as shown in FIG. 7B. The electronics unit carrier 714 can include magnets 716, such as for attracting opposing-polarity magnets located on the electronics unit received by the carrier 714, such as for helping to ensure good electrical contact between individual electrical contacts on the multi-contact connector block 706 and corresponding individual electrical contacts on the electronics unit being received into the carrier 714. The fabric 408 can provide a band to help securely hold the electronics unit against the carrier 714 and against the multi-contact connector block 706. The ends of the band formed by the fabric 408 can be adhesively or otherwise secured to the main body 104 portion of the fore portion 112 of the headband 100. FIG. 7D shows a view from the top, showing the receiving space for the electronics unit between the carrier 714 and the fabric 408 band.

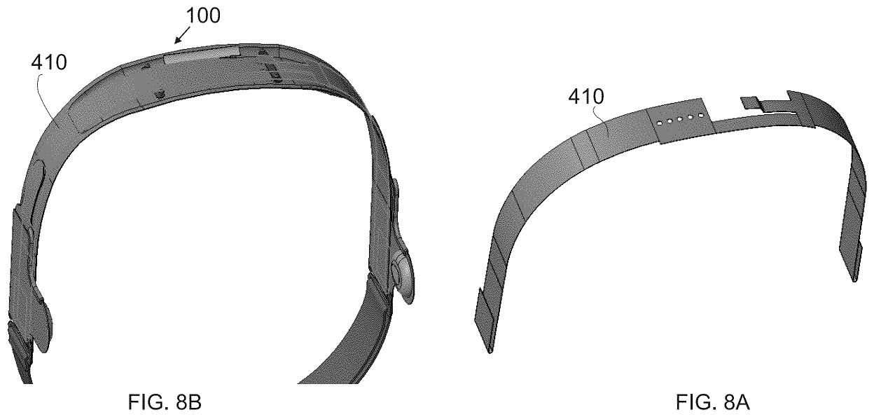

FIG. 8A shows a perspective view of an example of the FPC 410, which can include a unitary piece extending from FPC ends located near the temporal electrode assemblies 102A-B. FIG. 8B shows a cutaway view of portions of the headband 100 showing the FPC 410 located therein.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An EEG monitoring headband, comprising:
a human wearable headband, sized and shaped and otherwise configured to be worn encircling a head of a human subject at a location that is superior to eyes and ears of the subject, the headband including a pair of temporal electrodes protruding inferiorly from a main body portion of the headband and inwardly toward a central longitudinal axis defined through a center of the head of the subject in a superior-inferior direction, such that the temporal electrodes respectively contact skin of the subject at corresponding locations behind an ear of the subject.

2. The headband of claim 1, wherein the temporal electrodes are supported by respective pillows to inwardly locate respective working surfaces of the temporal electrodes closer to the central longitudinal axis of the subject than corresponding locations of the main body portion of the headband that are superior to and adjacent to the temporal electrodes.

3. The headband of claim 1, comprising:
pouches extending from the main body portion of the headband and protruding inferiorly from the main body portion of the headband to respectively carry corresponding temporal electrodes; and
resilient bent biasing members, sized and shaped and otherwise respectively configured to be insertable into corresponding pouches, the biasing members bent such that, when an individual one of the biasing members is inserted into an individual one of the pouches, an inferior portion of the individual one of the biasing members is located closer to the central longitudinal axis of the subject than a superior portion of the individual one of the biasing members, such that, when inserted into the respective pouches, the respective biasing members push respective individual ones of the temporal electrodes toward the central longitudinal axis of the subject.

4. The headband of claim 3, wherein the biasing members are provided in a kit including different bends provided in different individual ones of the biasing members to allow selection therebetween for selecting a desired amount of biasing of a corresponding temporal electrode toward the central longitudinal axis of the subject when a selected individual one of the biasing members is inserted into a corresponding individual one of the pouches.

5. The headband of claim 1, wherein the main body portion of the headband comprises:
a fore portion of the headband, sized and shaped and otherwise configured for extending along a forehead region of the subject and flexibly bending to extend along each temporal region of the subject to respective fore headband ends located beyond the temporal electrodes; and
an aft portion of the headband, sized and shaped and otherwise configured for extending along a rear skull region of the subject and flexibly bending to extend toward respective aft headband ends that are configured to engage with the respective fore headband ends.

6. The headband of claim 5, comprising:
an engagement clip, located between a fore headband end and a corresponding aft headband ends, the engagement clip permitting loop-back of a loop-back portion of the fore headband about at least a portion of the clip and toward an anterior direction of the subject to permit size adjustment of the fore portion of the headband.

7. The headband of claim 6, wherein a loop-back portion of the fore headband includes a fastener configured to secure the loop-back portion of the fore headband to a non-loop-back portion of the fore headband.

8. The headband of claim 7, wherein the fastener includes one of a hook or loop fabric and the non-loop-back portion of the fore headband includes an other one of the hook or loop fabric.

9. The headband of claim 6, wherein pouches, respectively carrying temporal electrodes, are respectively included in corresponding ones of the loop-back portions of the fore headband.

10. The headband of claim 6, comprising respective ear rails included in corresponding ones of the loop-back portions of the fore portion of the headband to respectively seat upon or behind an ear of the subject, or both.

11. The headband of claim 5, wherein the aft portion of the headband is adjustable length.

12. The headband of claim 11, wherein the aft headband is adjustable length including by being elastically stretchable.

13. The headband of claim 11, wherein the aft headband is adjustable length including by having a loop-back portion having a user-adjustable loop-back loop size to adjust a length of the aft headband.

14. The headband of claim 5, wherein the fore portion of the headband comprises a unitary flexible printed circuit (FPC) sized and shaped and otherwise configured for extending along a forehead region of the subject and flexibly bending to extend along each temporal region of the subject to respective FPC ends located at or near the ears of the subject, the FPC including respective electrically conductive traces from each of the temporal electrodes.

15. The headband of claim 1, comprising an electronics unit cradle assembly, located on a forehead portion of the headband, the electronics unit cradle assembly including electrical traces for receiving electrical signals from the temporal electrodes.

16. The headband of claim 15, wherein the electronics unit cradle assembly includes a pouch for receiving and carrying an electronics unit for signal-processing the electrical signals from the temporal electrodes.

17. A human wearable EEG monitoring headband to be worn encircling a head of a human subject at a location that is superior to eyes and ears of the subject, the headband comprising:
a fore portion of the headband, sized and shaped and otherwise configured for extending along a forehead region of the subject and flexibly bending to extend along each temporal region of the subject to respective fore headband ends, the fore portion of the headband including a pair of temporal electrodes respectively protruding inferiorly from a main body portion of the headband and inwardly toward a central longitudinal axis defined through a center of the head of the subject in a superior-inferior direction, such that the temporal electrodes respectively contact skin of the subject at corresponding locations behind an ear of the subject; and
an aft portion of the headband, sized and shaped and otherwise configured for extending along a rear skull region of the subject and flexibly bending to extend toward respective aft headband ends that are configured to engage with the respective fore headband ends via respective clips permitting loop-back of a loop-back portion of the fore headband about at least a portion of an individual one of the clips and toward an anterior direction of the subject to permit size adjustment of the fore portion of the headband; and
wherein the temporal electrodes respectively include corresponding pouches and corresponding bent stiffeners respectively insertable into individual ones of the pouches to at least one of position or bias a working surface of the temporal electrode against skin at a temporal of the subject.

18. The headband of claim 17, wherein the fore portion of the headband comprises a unitary flexible printed circuit (FPC) sized and shaped and otherwise configured for extending along a forehead region of the subject and flexibly bending to extend along each temporal region of the subject to respective FPC ends located at or near the ears of the subject, the FPC including respective electrically conductive traces from each of the temporal electrodes to an electronics unit carrier or cradle assembly located in a middle portion of the fore portion of the headband against a forehead of the subject.

19. A method of using a human wearable EEG monitoring headband, the method comprising:
locating the headband encircling a head of a human subject at a location that is superior to eyes and ears of the subject, wherein the headband includes:
a fore portion sized and shaped and otherwise configured for extending along a forehead region of the subject and flexibly bending to extend along each temporal region of the subject to respective fore headband ends, the fore portion of the headband including a pair of temporal electrodes respectively protruding inferiorly from a main body portion of the headband and inwardly toward a central longitudinal axis defined through a center of the head of the subject in a superior-inferior direction, such that the temporal electrodes respectively contact skin of the subject at corresponding locations behind an ear of the subject; and
an aft portion of the headband, sized and shaped and otherwise configured for extending along a rear skull region of the subject and flexibly bending to extend toward respective aft headband ends that are configured to engage with the respective fore headband ends via respective clips permitting loop-back of a loop-back portion of the fore headband about at least a portion of an individual one of the clips and toward an anterior direction of the subject to permit size adjustment of the fore portion of the headband; and
permitting one-handed user-adjustment of a length of the fore portion of the headband by detaching a loop-back portion of the headband, and at least one of pulling the loop-back portion of the headband in an anterior direction to tighten the fore portion of the headband or introducing slack into the loop-back portion of the headband in a posterior direction to loosen the fore portion of the headband.

20. The method of claim 19, further comprising adjusting a biasing of one or more of the temporal electrodes toward the central longitudinal axis of the subject by at least one of inserting or bending a biasing member received in a pouch carrying an individual one of the temporal electrodes.
